(19) United States
(12) Patent Application Publication
(10) Pub. No.: US 2015/0283335 A1
(43) Pub. Date: Oct. 8, 2015

(54) WATERPROOF INDICATOR AND METHOD OF USE THEREOF

(71) Applicant: Medtronic MiniMed, Inc., Northridge, CA (US)

(72) Inventor: Mark Lin, Northridge, CA (US)

(73) Assignee: Medtronic MiniMed, Inc., Northridge, CA (US)

(21) Appl. No.: 14/247,031

(22) Filed: Apr. 7, 2014

(51) Int. Cl. A61M 5/50; A61M 5/142

(52) U.S. Cl. CPC A61M 5/5086; A61M 5/14244

(57) ABSTRACT

A medical device having an indicator to indicate whether the medical device has a waterproof seal enclosing an interior volume. The medical device includes a housing having an indicator formed on the exterior surface of the housing. The indicator is capable of moving from a first position to a second position, where the first position indicates that the medical device housing has a waterproof seal to prevent ingress of water into the interior volume of the medical device and the second position indicates that there is a leak in the housing.

### FIELD OF THE INVENTION

This invention relates to leak indicators and methods, and in particular embodiments, an indicator built into a medical device, such as an infusion pump, to detect and indicate whether the medical device has a waterproof seal.

### BACKGROUND OF THE INVENTION

Medical devices typically contain sensitive components, such as electrical circuit boards and the like, that require waterproof protection. Furthermore, many portable medical devices are worn continuously on the body for hours to days to monitor or provide treatment to the body. It is important that such devices are waterproof or water resistant if worn while swimming, bathing, showering, or the like.

During the manufacturing process, medical devices are often sealed to enclose the sensitive components and provide the needed water protection. The devices can be tested and be given a rating based on the level of waterproof protection that is provided. Current methods of testing whether the seal of an object is waterproof involve water immersion tests. For example, an ingress protection (IP) rating is an industry standard for waterproof performance. An IPX8 rating indicates that a device is water-tight and suitable for continuous submersion in water under conditions specified by the manufacturer, for example, by immersing the device under water for thirty minutes at a depth of eight feet (2.4 meters) without any water ingress. An IPX7 rating can indicate that a device is protected against water immersion for thirty minutes at a depth of up to one meter. The water immersion tests require time, resources, and can be cumbersome to perform.

### BRIEF SUMMARY OF THE INVENTION

Embodiments of the present invention include leak and/or waterproof indicators and detection methods for providing simple visual or sensory methods of determining whether a medical device has a waterproof seal, which obviate for practical purposes, the above mentioned limitations.

According to an embodiment of the invention, a medical device includes an indicator to indicate whether the medical device is sealed. In particular embodiments, the medical device can be an infusion pump or a transmitter. The medical device includes a housing having an interior volume and an exterior surface. The indicator can be formed on the exterior surface of the medical device housing. In embodiments, the exterior surface of the housing is rigid and the indicator is flexible. The indicator is capable of moving from a first position to a second position. The first position can indicate that the medical device housing has a seal to prevent ingress into the interior volume of the medical device. The seal may prevent ingress of any material such as air, water, contaminants, or the like. For example, in embodiments the first position can indicate the medical device housing has a waterproof seal to prevent water from entering into the interior of the medical device. The second position can indicate that there is a leak in the housing and thus the seal of the housing is broken.

In further embodiments, the first position of the indicator is a depressed or recessed position that protrudes inward toward the interior volume of the housing and the second position of the indicator is a raised position that protrudes outward from the exterior surface of the medical device housing and away from the interior volume of the medical device housing. In yet a further embodiment, the second position is a flat position that is flush with the exterior surface of the housing. In another embodiment, the first position is flush with the exterior surface of the housing, and the second position protrudes outward from the exterior surface of the medical device housing and away from the interior volume of the medical device housing. In another embodiment, the first position is flush with the exterior surface of the housing, and the second position protrudes inward toward the interior volume of the housing. In yet another embodiment, the first position protrudes outward from the exterior surface of the medical device housing and away from the interior volume of the medical device housing, and the second position protrudes inward toward the interior volume of the housing. In another embodiment, the first position protrudes outward from the exterior surface of the medical device housing and away from the interior volume of the medical device housing, and the second position is flush with the exterior surface of the housing.

In further embodiments, the indicator further includes at least one tactile element. In such embodiments, one or more tactile elements can protrude from the indicator when the indicator is in the second position, providing touch and visual indications of whether the medical device housing is sealed. In another embodiment, the indicator further includes a first pattern displayed in the first position and a second pattern displayed in the second position, providing a further visual indication of whether the medical device housing is sealed. The first and/or second patterns can include color, text and/or at least one symbol.

In a particular embodiment, the medical device housing further includes a rigid portion beneath the flexible indicator. The top surface of the portion of the medical device housing beneath the indicator includes a color, pattern, symbol, text, or a combination thereof. In such embodiments, the indicator is translucent such that the color, pattern, symbol and/or text on the top surface of the portion of the medical device housing beneath the indicator is visible through the indicator only when the indicator is in a position that contacts the top surface of the portion of the housing beneath the indicator.

In yet another embodiment, the medical device housing further includes a first electrical contact and a second electrical contact. In such embodiments, the indicator further includes a conductive material. The electrical contacts can indicate the position of the indicator because the indicator contacts the first and second electrical contacts only when the indicator is in a position that protrudes inward toward the interior volume of the housing. The indicator will not come in contact with the electrical contacts when the indicator is in a raised or flat position. In alternative embodiments, the indicator may contact the electrical contacts when the indicator is in a flat position, but will not contact the electrical contacts in a raised position.

In further embodiments, the indicator can provide a sound when the indicator moves from the first position to the second position, providing an auditory indication of whether the medical device housing is sealed.

The indicator and/or housing can be formed using suitable manufacturing methods including, but not limited to machining, ultrasonic welding, overmolding, injection molding, vacuum forming, blow molding, adhesive bonding or joining, and 3-D printing. The indicator and/or housing can further be made of a plastic material or other suitable material.

Various methods for determining if a medical device housing has a waterproof seal can be ascertained from the description of embodiments of the invention herein. In one embodiment, the method comprises providing a medical device housing having an interior volume, an exterior surface, and an indicator on the exterior surface. The indicator is moveable from a first position to a second position and vice versa. In the first step, the indicator can be provided in the second position. The second step of the method can include sealing the medical device housing in a low pressure environment. In this sealing step, the indicator can remain in the second position. The next step involves returning the medical device housing to a normal atmospheric environment such that the indicator moves to the first position. The indicator remains in the first position so long as the seal is intact. The indicator moves to the second position when there is a leak in the housing. In embodiments, the step of providing the medical device can further include: overmolding the indicator on the exterior surface of the housing; ultrasonically welding the indicator on the exterior surface of the housing; adhesively bonding the indicator on the exterior surface of the housing; vacuum forming the indicator on the exterior surface of the housing; blow molding the indicator on the exterior surface of the housing; forming the indicator on the exterior surface of the housing using injection molding; or forming the indicator on the exterior surface of the housing using 3-D printing.

Another embodiment of a method for determining whether a medical device housing is sealed comprises the steps of: providing a sealed medical device housing having an interior volume, an exterior surface, and an indicator on the exterior surface, where the indicator is moveable from a first position to a second position and the indicator is in the first position; and observing the position of the indicator to determine whether the medical device housing is sealed, wherein the first position indicates that the medical device housing is sealed and the second position indicates that there is a leak in the medical device housing. The step of providing the sealed medical device housing can further include sealing the medical device housing in low pressure, high pressure, low temperature or high temperature environments.

Other features and advantages of the invention will become apparent from the following detailed description, taken in conjunction with the accompanying drawings which illustrate, by way of example, various features of embodiments of the invention.

### BRIEF DESCRIPTION OF SEVERAL VIEWS OF THE DRAWINGS

A detailed description of embodiments of the invention will be made with reference to the accompanying drawings, wherein like numerals designate corresponding parts in the several figures.

FIG. 1 is top perspective view of a medical device having a recessed indicator in accordance with an embodiment of the present invention.

FIG. 2 is top perspective view of a medical device having a protruding indicator in accordance with an embodiment of the present invention.

FIG. 3 is top orthogonal view of a medical device having an indicator in accordance with an embodiment of the present invention.

FIG. 4 is a side cross-sectional view along A-A of the medical device of FIG. 3 having an indicator indicating a sealed housing in accordance with an embodiment of the present invention.

FIG. 5 is a side cross-sectional view along A-A of the medical device of FIG. 3 having an indicator indicating an unsealed medical device in accordance with an embodiment of the present invention.

FIG. 6 is a side cross-sectional view of a medical device having an indicator indicating a sealed medical device in accordance with an embodiment of the present invention.

FIG. 7 is a side cross-sectional view of a medical device having an indicator indicating an unsealed medical device in accordance with an embodiment of the present invention.

FIG. 8 is a side cross-sectional view of a medical device having an indicator indicating an unsealed medical device in accordance with an embodiment of the present invention.

FIGS. 9A and 9B are flowcharts that illustrate embodiments of the position of an indicator of a medical device before, during, and after sealing the medical device housing under low pressure in accordance with embodiments of the present invention.

FIGS. 10A and 10B are flowcharts that illustrate embodiments of the position of an indicator of a medical device before, during, and after sealing the medical device housing in a high temperature environment in accordance with embodiments of the present invention.

FIGS. 11A and 11B are flowcharts that illustrate embodiments of the position of an indicator of a medical device before, during, and after sealing the medical device housing under high pressure in accordance with embodiments of the present invention.

FIGS. 12A and 12B are flowcharts that illustrate embodiments of the position of an indicator of a medical device before, during, and after sealing the medical device housing in a low temperature environment in accordance with embodiments of the present invention.

FIGS. 13A and 13B are a side cross-sectional view and a top view, respectively, of an indicator in a recessed position and having a first pattern in accordance with an embodiment of the present invention.

FIGS. 13C and 13D are a side cross-sectional view and a top view, respectively, of the indicator of FIGS. 13A and 13B, the indicator in a raised position and having a second pattern in accordance with an embodiment of the present invention.

FIGS. 14A and 14B are a side cross-sectional view and a top view, respectively, of an indicator in a recessed position and including tactile elements in accordance with an embodiment of the present invention.

FIGS. 14C and 14D are a side cross-sectional view and a top view, respectively, of the indicator of FIGS. 14A and 14B, the indicator in a raised position and including tactile elements in accordance with an embodiment of the present invention.

FIGS. 15A and 15B are a side cross-sectional view and a top view, respectively, of an indicator in a recessed position and including text on a portion of the housing in accordance with an embodiment of the present invention.

FIGS. 15C and 15D are a side cross-sectional view and a top view, respectively, of the indicator of FIGS. 15A and 15B, the indicator in a raised position in accordance with an embodiment of the present invention.

FIGS. 16A and 16B are a side cross-sectional view and a top view, respectively, of an indicator in a raised position in accordance with an embodiment of the present invention.

FIGS. 16C and 16D are a side cross-sectional view and a top view, respectively, of the indicator of FIGS. 16A and 16B, of an indicator in a recessed position and including a symbol on a portion of the housing in accordance with an embodiment of the present invention.

FIGS. 17A and 17B are side cross-sectional views of a conductive indicator in a recessed position and raised position, respectively, in accordance with an embodiment of the present invention.

FIG. 18 is a perspective view of an infusion pump having an indicator in a recessed position in accordance with an embodiment of the present invention.

FIG. 19 is a perspective view of an infusion pump having an indicator in a raised position in accordance with an embodiment of the present invention.

FIG. 20 is a side cross-sectional view of a sensor and a transmitter with an indicator in accordance with an embodiment of the present invention.

FIG. 21 is a top view of a sensor and a transmitter with an indicator in accordance with an embodiment of the present invention.

### DETAILED DESCRIPTION OF THE INVENTION

As shown in the drawings for purposes of illustration, the invention is embodied in a medical device incorporating an indicator to detect and indicate whether there is a leakage path between the interior of the medical device and the outside environment. The following detailed description is merely illustrative in nature and is not intended to limit the embodiments of the subject matter or the application and uses of such embodiments. While the subject matter described herein can be implemented with any electronic device, exemplary embodiments described below are implemented in the form of medical devices, such as portable electronic medical devices.

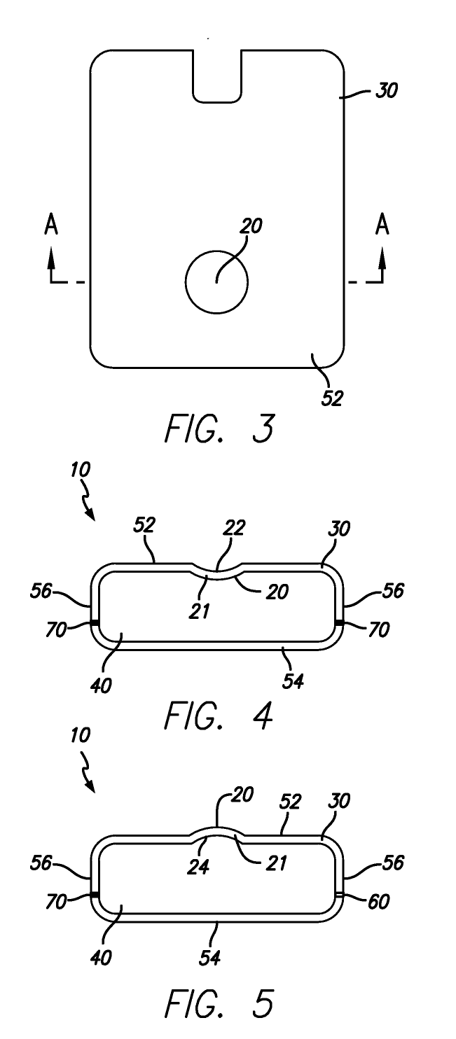

According to embodiments of the invention generally shown in FIGS. 1-5, a medical device 10 having a housing 30 with a waterproof indicator 20 is provided. The medical device housing 30 has an exterior surface 50 and an interior volume 40 to hold the components of the medical device. In the embodiments shown, the exterior surface 50 can have multiple sides, including a top exterior surface 52, side exterior surfaces 56, and a bottom exterior surface 54. In some embodiments, the housing 30 can further be comprised of one or more assembled portions. The portions of the device 10 can be connected using an adhesive, glue, ultrasonic welding or the like prior to sealing the portions together. In embodiments, the medical device 10 may have a seam 70 where the one or more portions are joined to one another. Medical devices 10 that include more than one sealed compartments can utilize an indicator 20 for each separate compartment.

The indicator 20 can be incorporated in any medical device 10 that includes a sealed housing 30 enclosing one or more components (not shown) in the interior volume 40 that require protection from environmental factors, including but not limited to waterproof protection. In preferred embodiments of the invention, the indicator 20 is built into one or more of any of the exterior surfaces 50 of the medical device housing 30. The indicator 20 is moveable from a first position 22 to a second position 24. The first position 22 indicates that the housing 30 has a waterproof seal. The second position 24 indicates that there is a leak 60 in the housing 30 that has broken the seal. If and when the waterproof seal of the device 10 is broken, the indicator 20 will move from a first position 22 to a second position 24.

In embodiments, the indicator 20 is a button, tab, flexible membrane, or the like. The indicator 20 can be positioned on a top 52, bottom 54, or side 56 exterior surface 50 of the housing 30. Generally shown in FIGS. 1-3, an embodiment of the device 10 incorporates the indicator 20 in a top exterior surface 52 of the housing 30. Though the indicator 20 is circular in the embodiments shown, the indicator 20 can be formed in any shape such as a square, rectangle, diamond, star or the like.

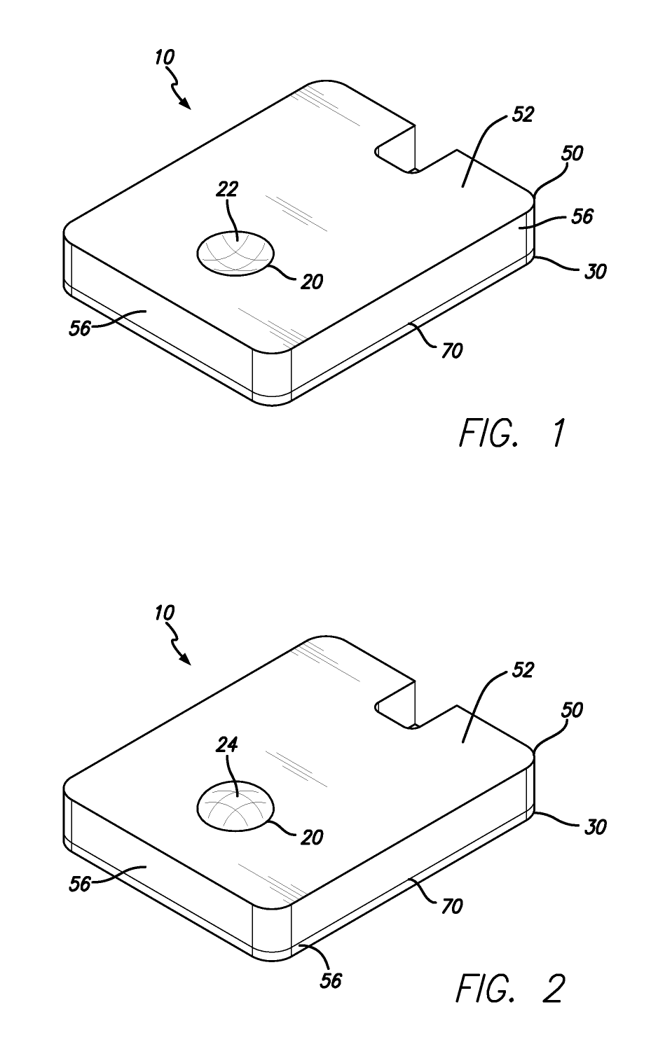

In the particular embodiment shown in FIGS. 1 and 4, the indicator 20 is shown as a detent in the housing 30, having a recessed or depressed first position 22 that protrudes inward toward the interior volume 40 of the housing 30. In embodiments, so long as the indicator 20 remains in the first position 22, the indicator 20 indicates that the seal of the housing 30 remains intact and the device 10 is waterproof. In some embodiments, the indicator 20 can indicate a waterproof rating of up to and including IPX8 for the medical device. In the particular embodiment shown in FIGS. 2 and 5, the indicator 20 is shown having a raised second position 24. The raised second position 24 of the indicator 20 indicates that the housing 30 has a leak 60 and has lost its waterproof seal. Though the embodiment shown in FIG. 5 shows a leak 60 in a seam 70 of the housing 30, the leak 60 can be located at any position within the housing 30, as shown in an embodiment in FIG. 7. Accordingly, the indicator 20 provides a visual or sensory method to determine if there is a leak, opening, break, crack, fissure, fracture, breach or the like in the medical device housing 30 that could allow air or water to enter into the interior volume 40 of the housing 30.

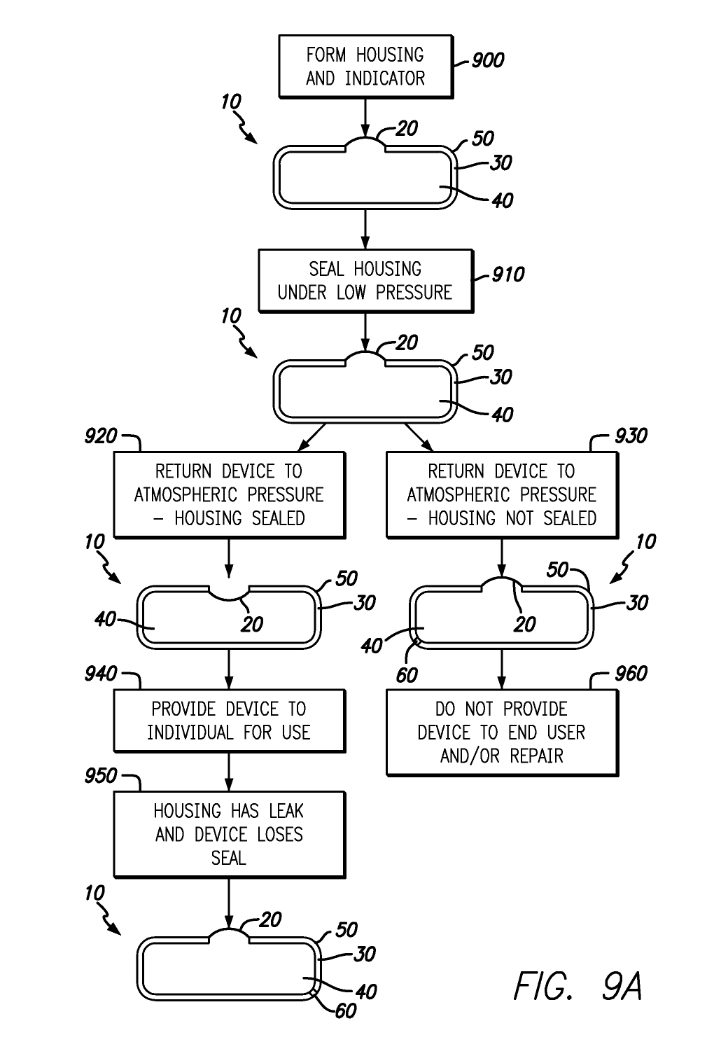

In alternative embodiments, the first position 22 or second position 24 of the indicator 20 can be a flat position that is flush with, or lies in the same plane of the exterior surface 50 of the housing 30. A non-limiting example of an indicator 20 in a flat position is shown in FIG. 8.

In yet alternative embodiments, the first position 22 of the indicator 20 can be a raised position. In further embodiments, the second position 24 of the indicator 20 can be a recessed position. Depending on the method of sealing the housing 30 of the device 10, different embodiments of the first 22 and second 24 positions of the indicator 20 include: a recessed first position 22 and a raised second position 24; a raised first position 22 and a recessed second position 24; a flat first position 22 and a raised second position 24; a flat first position 22 and a recessed second position 24; a raised first position 22 and a flat second position 24; and a recessed first position 22 and a flat second position 24.

The indicator 20 can be made of the same or different material as the housing 30. In embodiments, the medical device housing 30 and/or the indicator 20 can comprise plastic, polymeric, and/or thermoplastic materials including but not limited to polycarbonate (PC), polyester (PES), polyethylene terephthalate (PET), polycarbonate/polyethylene terephthalate (PC/PET), polybutylene terephthalate (PBT), polycarbonate/polybutylene terephthalate (PC/PBT), polyethylene (PE), high-density polyethylene (HDPE), low-density polyethylene (LDPE), polyvinyl chloride (PVC), polypropylene (PP), polystyrene (PS), high impact polystyrene (HIPS), acrylonitrile butadiene styrene (ABS), polycarbonate/acrylonitrile butadiene styrene (PC/ABS), polyurethanes (PU), polyamides (PA), or like materials suitable for forming a sealed durable housing 30 and/or a moveable indicator 20.

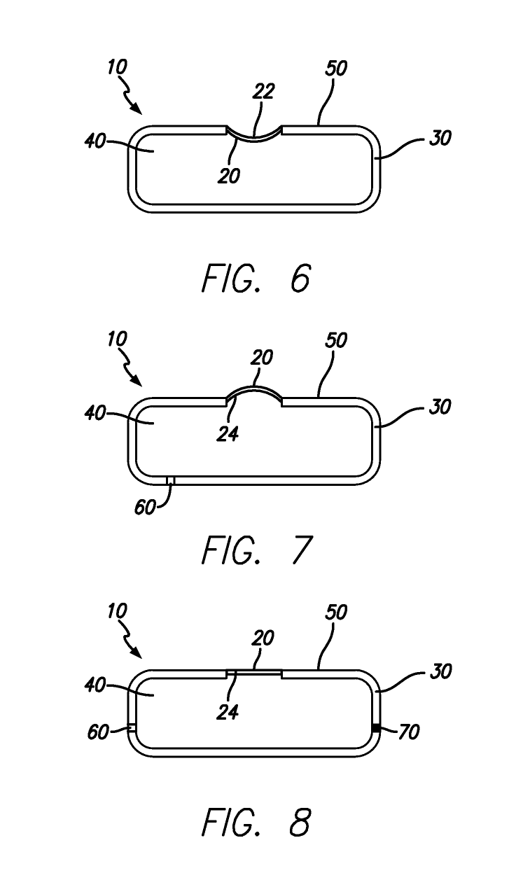

In preferred embodiments, the housing 30 is rigid and the indicator 20 is flexible. In embodiments where the indicator 20 is made of the same material as the medical device housing 30, the body 21 of the indicator 20 can have a reduced thickness or thinner diameter than that of the housing 30 to provide the flexibility needed to allow the indicator 20 to move from a first position 22 to a second position 24. Embodiments showing the indicator 20 having a reduced thickness compared to that of the housing 30 are shown in FIGS. 6-8. In alternative embodiments shown in FIGS. 4 and 5, the indicator 20 and housing 30 have the same diameter or uniform thickness.

In alternative embodiments, the indicator 20 comprises a different plastic material than that of the housing 30, or another suitable flexible material. For example, the indicator 20 can be made of a metal and comprise a thin curved metal disc that is overmolded onto the housing 30. In embodiments where the housing 30 and indicator 20 are made of different materials, the indicator 20 can have the same or a different thickness than the housing 30.

Embodiments of the indicator 20 can be formed in the housing 30 during a method of manufacture or can be added to the housing 30 during the method of manufacture. Thus, the indicator 20 and housing 30 can form one integrated unit or may be two separate components attached with one another. Methods of forming the indicator 20 and/or medical device housing 30 include injection molding, vacuum forming, blow molding, 3-D printing or other suitable method of manufacture. In alternative embodiments, the indicator 20 can be 2-shot overmolded on the housing 30, ultrasonically welded on the housing 30, or adhesively bonded to the housing 30. In further embodiments, a combination of two or more of the aforementioned methods can be used to manufacture the indicator and/or housing.

In some embodiments, the medical device 10 may have one or more portions of the housing 30 that are joined during the sealing process. In such embodiments, the juncture between the one or more portions can create a seam 70 within the housing 30, as shown in FIGS. 1 and 2. Indicators 20 and methods for testing for leaks 60 in the medical device housing 30 can include detection at the seams 70 in addition to leaks 60 at any other location through the medical device housing 30, as shown in the embodiment in FIG. 7. In embodiments, the components of the device 10 and/or portions of the device housing 30 can be assembled or connected using an adhesive, glue, mechanical joining, ultrasonic welding, or the like prior to sealing the portions together.

In embodiments, once the device housing 30 having an indicator 20 is formed, the medical device housing 30 is sealed to provide the waterproof protection. In alternative embodiments, the medical device housing 30 is sealed when it is formed.

In embodiments, the indicator 20 is activated or set in an active, detecting mode during the sealing process of the housing 30. Different methods of sealing can be employed to set the indicator 20 in a working mode. In one embodiment, the medical device housing 30 is sealed under low pressure in a low pressure environment by the use of a vacuum pump. For example, a vacuum may be used to draw air out of a closed environment where the device will be sealed, such as a chamber of any suitable size. In embodiments, the air will be pulled out of the chamber to create a sealing environment less than 1 atm or 14.7 psi. In some embodiments that utilize adhesive to connect portions of the housing 30, the sealing process can be performed when the adhesive is cured. Prior to the application of the low pressure vacuum, the indicator 20 is naturally flat or protrudes from an exterior surface 50 of the housing 30. After the housing 30 is sealed and is returned to a normal atmospheric pressure environment, the indicator 20 is drawn in towards the interior volume 40.

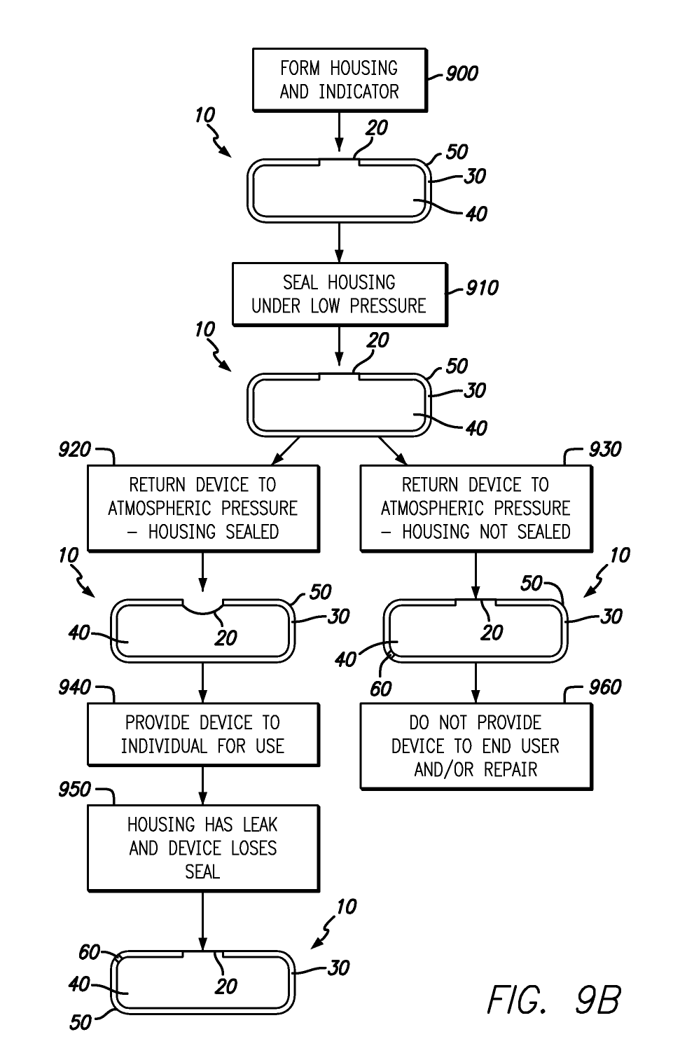

Embodiments of the position of the indicator 20 before, during, and after the sealing process in a low pressure environment are illustrated in the exemplary flow charts of FIGS. 9A and 9B. In the first step, prior to the sealing process of the device, the indicator 20 is initially provided or formed in a raised position that is protruding from an exterior surface 50 of the housing 30 (step 900). In alternative embodiments shown in FIG. 9B, the indicator 20 is flush with the housing 30 so that it is in a flat position (step 900). Next, the housing 30 is sealed under low pressure and the indicator 20 remains in the raised position or flat position, respectively, during the sealing process in a low pressure environment (step 910). Upon returning the device 10 to a normal atmospheric environment, if the housing 30 is properly sealed (step 920), the indicator 20 is drawn inward because of the pressure differential between the interior volume 40 and the outside of the housing. Thus, the recessed position indicates that the housing 30 is properly sealed. However, if there is a leak 60 in the device housing 30, the inner and outer pressure will equilibrate, causing the indicator 20 to remain in the initial raised or flat position, and thus indicating that the device housing 30 is not sealed (step 930). If the housing 30 was not properly sealed, the device 10 may not be provided to the end user (step 960). If the housing 30 was properly sealed (step 920), the manufacturer may provide the device 10 to an end user (step 940). If the sealed housing 30 later loses its seal or has a leak 60, then interior volume 40 pressure and the exterior pressure will equilibrate, and the indicator 20 will move from the first recessed position to a second raised position (step 950). In an alternative embodiment, after the sealed housing 30 loses its seal, the indicator 20 can move from the first recessed position to a second flat position (shown in FIG. 9B). In cases where the device housing 30 loses its seal, the end user can return the device 10 to the manufacturer for repair, for example, if the device is under warranty.

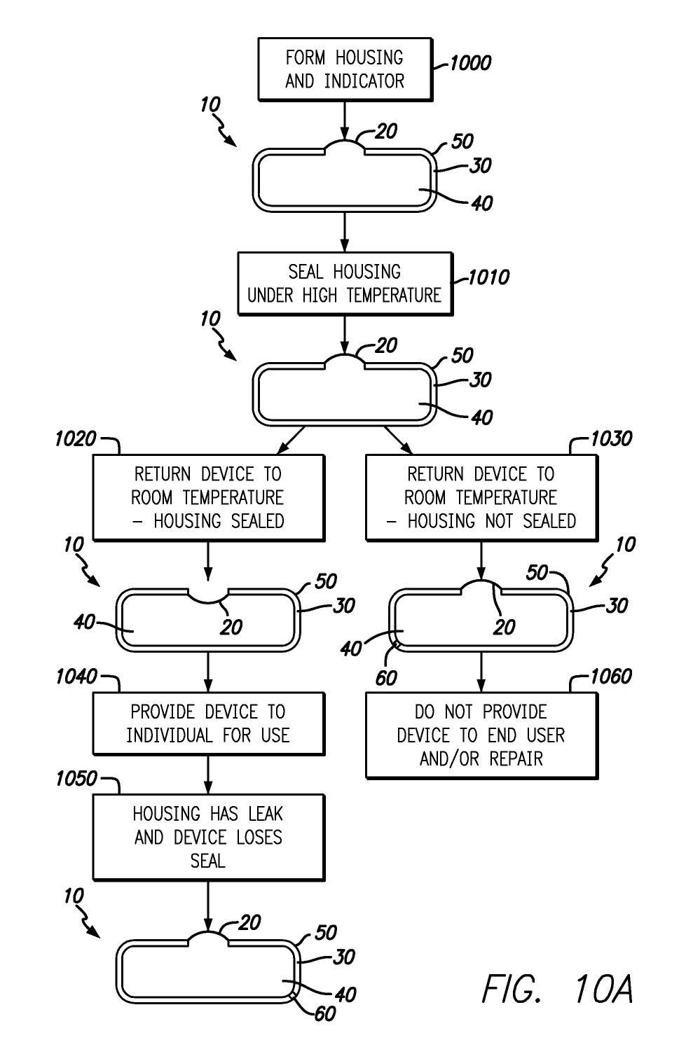

In yet alternative embodiments, the sealing process can be done in a hot environment. Upon cooling the device 10 to room temperature, an indicator 20 in a flat or raised position would be drawn inward toward the interior volume 40 of the housing 30, to a first recessed position 22. If the device 10 loses its seal, the indicator 20 will move to a second, raised or flat position 24 to indicate the housing 30 is no longer sealed. Embodiments of the position of the indicator 20 before, during, and after the high temperature sealing process are illustrated in the exemplary flow charts of FIGS. 10A and 10B. In the first step, prior to the sealing process of the device, the indicator 20 is initially formed or provided in a raised position that is protruding from an exterior surface 50 of the housing 30 (step 1000). In alternative embodiments shown in FIG. 10B, the indicator 20 is flush with the housing 30 so that it is in a flat position (step 1000). Next, the housing 30 is sealed under high temperature and the indicator 20 remains in the raised position or flat position, respectively, during the sealing process in a high temperature environment (step 1010). Upon returning the device 10 to a room temperature environment, if the housing 30 is properly sealed (step 1020), the indicator 20 is drawn inward as the temperature is lowered because of the ideal gas law (PV=nRT), which governs the relationship between the pressure of the interior volume 40 and the temperature. Thus, the recessed position indicates that the housing 30 is properly sealed. However, if there is a leak 60 in the device housing 30, the indicator 20 will remain in the initial raised or flat position upon returning the device 10 to room temperature, and thus indicate that the device housing 30 is not sealed (step 1030). If the housing 30 was not properly sealed, the device 10 may not be provided to the end user (step 1060). If the housing 30 was properly sealed (step 1020), the manufacturer may provide the device 10 to an end user (step 1040). If the sealed housing 30 later loses its seal or has a leak 60, then interior volume 40 pressure and the exterior pressure will equilibrate, and the indicator 20 will move from the first recessed position to a second raised position (step 1050), or a second flat position (shown in FIG. 10B). In this case, the end user can return the device 10 to the manufacturer for repair, for example, if the device is under warranty.

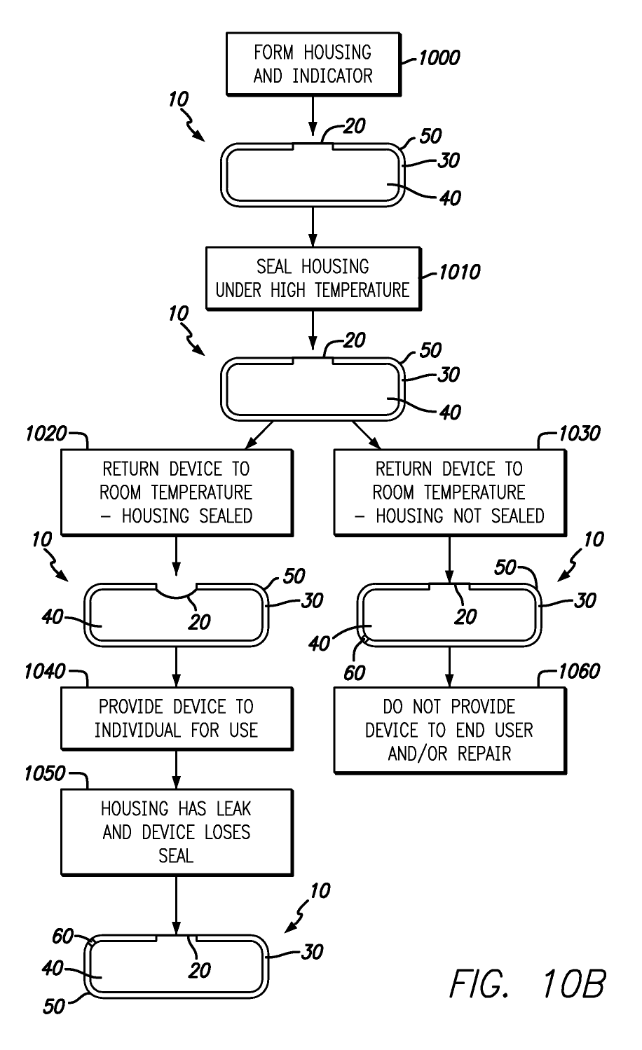

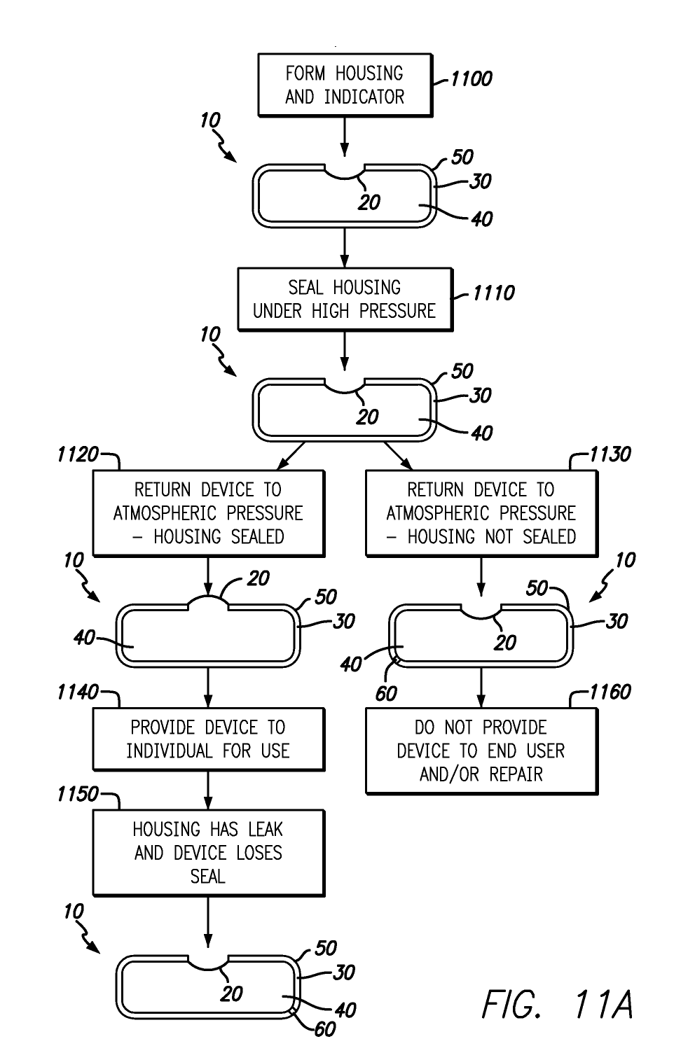

In further embodiments, the sealing process can be done under high pressure. For example, a compressed air source such as an air pump or pressure pump may be used to pump air into a closed environment where the device will be sealed, such as a chamber of any suitable size. In embodiments, the chamber will be pressurized to create a sealing environment greater than 1 atm or 14.7 psi. Embodiments of the position of the indicator 20 before, during, and after the high pressure sealing process are illustrated in the exemplary flow charts of FIGS. 11A and 11B. In the first step, prior to the sealing process of the device, the indicator 20 is initially provided or formed in a depressed position that is recessed from an exterior surface 50 of the housing 30 (step 1100). In alternative embodiments shown in FIG. 11B, the indicator 20 is flush with the housing 30 so that it is in a flat position (step 1100). Next, the housing 30 is sealed under high pressure and the indicator 20 remains in the depressed position or flat position, respectively, during the sealing process in a high pressure environment (step 1110). Upon returning the device 10 to a normal atmospheric pressure environment, if the housing 30 is properly sealed (step 1120), the indicator 20 is pushed outward because the pressure differential between the interior volume 40 and the outside of the housing has changed. Thus, the raised position indicates that the housing 30 is properly sealed. However, if there is a leak 60 in the device housing 30, the inner and outer pressure will equilibrate, causing the indicator 20 to remain in the initial depressed or flat position, and thus indicating that the device housing 30 is not sealed (step 1130). If the housing 30 was not properly sealed, the device 10 may not be provided to the end user (step 1160). If the housing 30 was properly sealed (step 1120), the manufacturer may provide the device 10 to an end user (step 1140). If the sealed housing 30 later loses its seal or has a leak 60, then interior volume 40 pressure and the exterior pressure will equilibrate, and the indicator 20 will move from the first raised position to a second recessed position (step 1150), or a flat position (shown in FIG. 11B). In this case, the end user can return the device 10 to the manufacturer for repair, for example, if the device is under warranty.

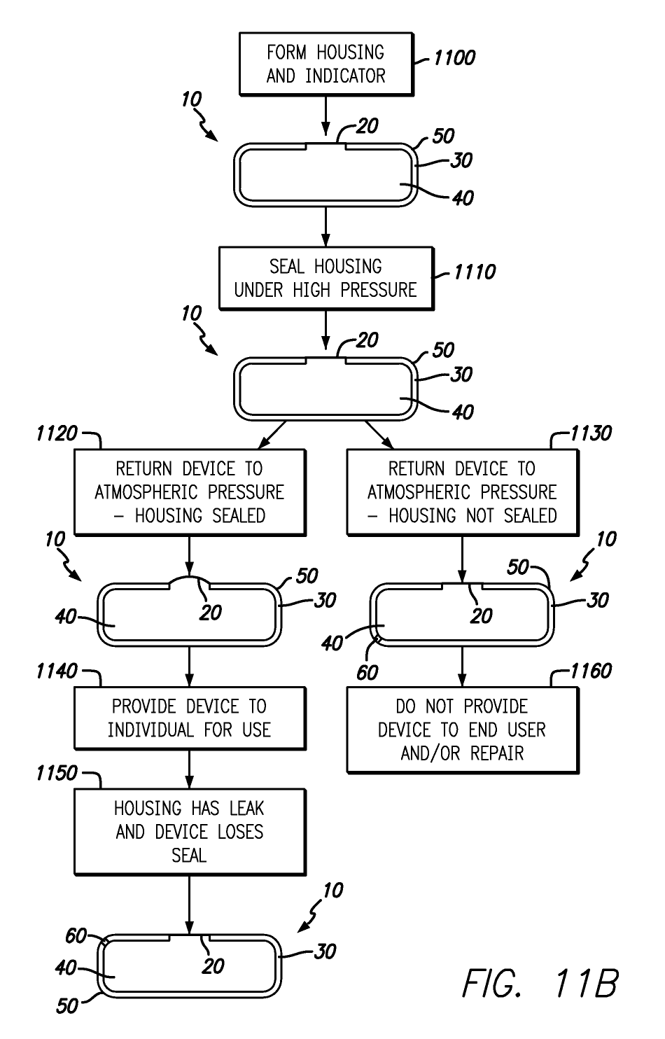

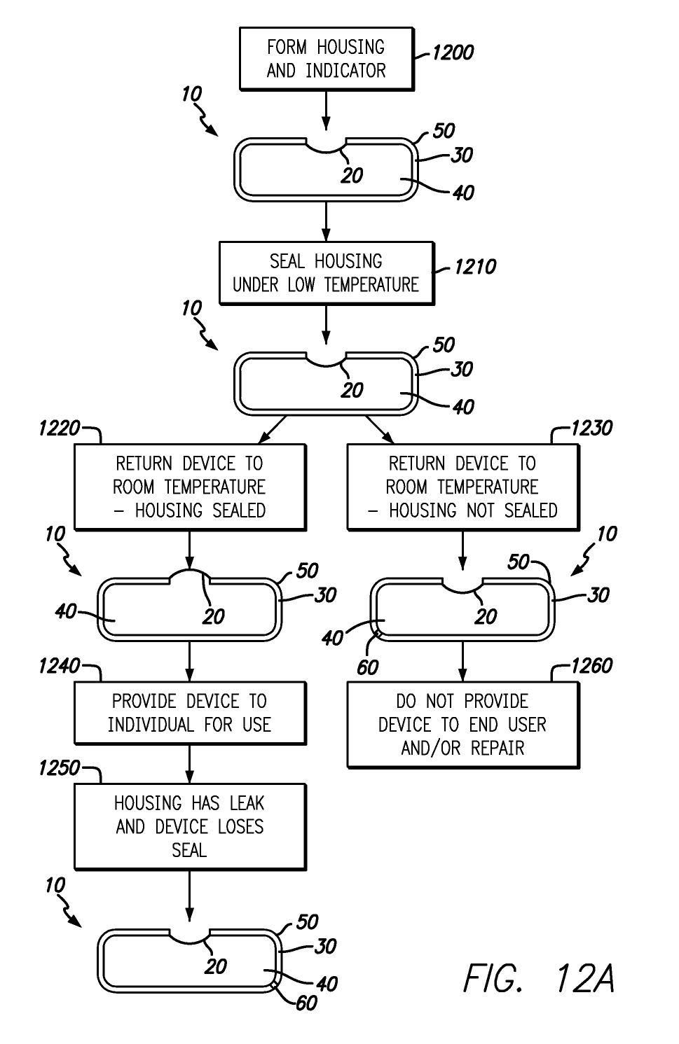

In yet further embodiments, the sealing process can be done in a low temperature environment. Embodiments of the position of the indicator 20 before, during, and after the low temperature sealing process are illustrated in the exemplary flow charts of FIGS. 12A and 12B. In the first step, prior to the sealing process of the device, the indicator 20 is initially formed or provided in a depressed position that is recessed from an exterior surface 50 of the housing 30 (step 1200). In alternative embodiments shown in FIG. 12B, the indicator 20 is flush with the housing 30 so that it is in a flat position (step 1200). Next, the housing 30 is sealed under low temperature and the indicator 20 remains in the depressed position or flat position, respectively, during the sealing process in a low temperature environment (step 1210). Upon returning the device 10 to room temperature (step 1220), if the housing 30 is properly sealed, the indicator 20 is pushed outward because of the ideal gas law (PV=nRT), which governs the relationship between the pressure of the interior volume 40 and the temperature. Thus, the raised or protruding position indicates that the housing 30 is properly sealed. However, if there is a leak 60 in the device housing 30, the indicator 20 will remain in the initial depressed or flat position upon returning the device 10 to room temperature, and thus indicate that the device housing 30 is not sealed (step 1230). If the housing 30 was not properly sealed, the device 10 may not be provided to the end user (step 1260). If the housing 30 was properly sealed (step 1220), the manufacturer may provide the device 10 to an end user (step 1240). If the sealed housing 30 later loses its seal or has a leak 60, then interior volume 40 pressure and the exterior pressure will equilibrate, and the indicator 20 will move from the first raised position to a second recessed position (step 1250) or a second flat position (shown in FIG. 12B). In this case, the end user can return the device 10 to the manufacturer for repair, for example, if the device is under warranty.

In some embodiments, the sealing process can occur in both a low pressure and high temperature environment. In other embodiments, the sealing process can occur in both a high pressure and low temperature environment.

In further embodiments shown in FIGS. 13A-13D, the indicator 20 can display a first pattern 26 in the first position 22 and a second pattern 28 in the second position 24. For example, as shown in embodiments in FIGS. 13A and 13B, the indicator 20 is in a recessed first position 22 and displays a solid first pattern 26. FIGS. 13C and 13D show the same indicator 20 in a raised second position 24 and displaying a striped second pattern 28. The patterns can change according to the shift of the indicator body 21 from a contracted, recessed position to an expanded, raised position, or vice versa. The first 26 and second 28 patterns can include any suitable design, text, symbol, and/or color. Embodiments utilizing changing patterns and/or colors displayed by the indicator 20 can provide further visual confirmation to determine whether or not the housing 30 is sealed.

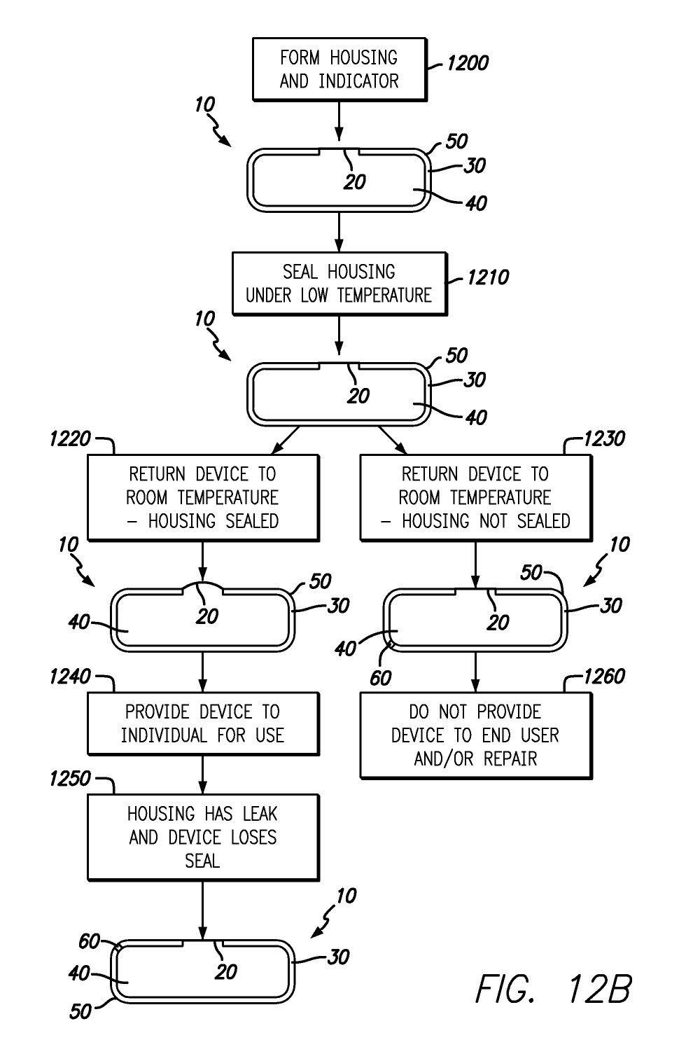

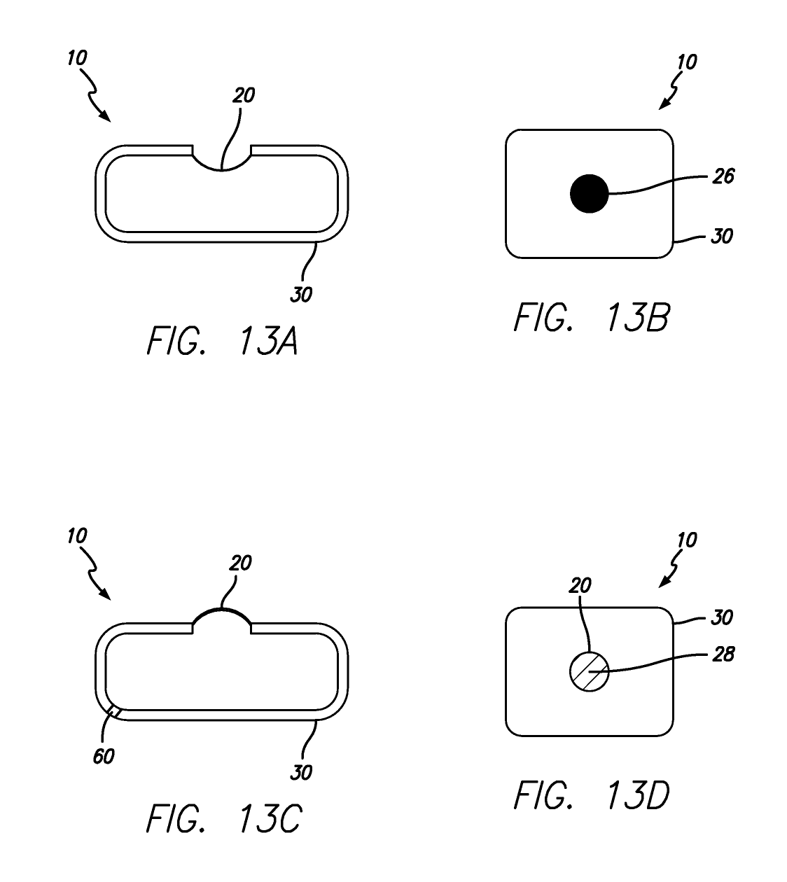

In alternative embodiments, tactile elements 80 are incorporated in the indicator 20 such that the indicator 20 is smooth to the touch in the first position 22 and rough to the touch in a second position 24. FIGS. 14A-14D illustrate embodiments of the indicator 20 including one or more tactile elements 80 incorporated in its body 21. For example, as shown in embodiments in FIGS. 14A and 14B, the indicator 20 is in a recessed first position 22 and is smooth to the touch. As shown in embodiments in FIGS. 14C and 14D, the indicator 20 is in a raised second position 24 and has tactile elements 80 protruding from the exterior surface of the indicator body 21. One or more tactile elements 80 can appear according to the shift of the indicator 20 from a contracted, recessed or depressed position to an expanded, raised position. For example, this shift may occur after a device 10 is sealed in a low pressure or high temperature environment to provide a first recessed position, which then shifts to a raised position once the device 10 loses its seal. In alternative embodiments, one or more tactile elements 80 can disappear according to the shift of the indicator 20 from an expanded raised position to a contracted, recessed or depressed position. For example, this shift may occur after a device 10 is sealed in a high pressure or low temperature environment to provide a first raised position, which then shifts to a recessed position once the device 10 loses its seal. The tactile elements 80 can comprise any suitable structure protruding from the outer surface of the indicator 20. In addition to having a recessed, flat, or raised position, the tactile elements 80 can assist individuals with impaired vision to feel the position of the indicator 20 to determine if the medical device housing 10 is sealed.

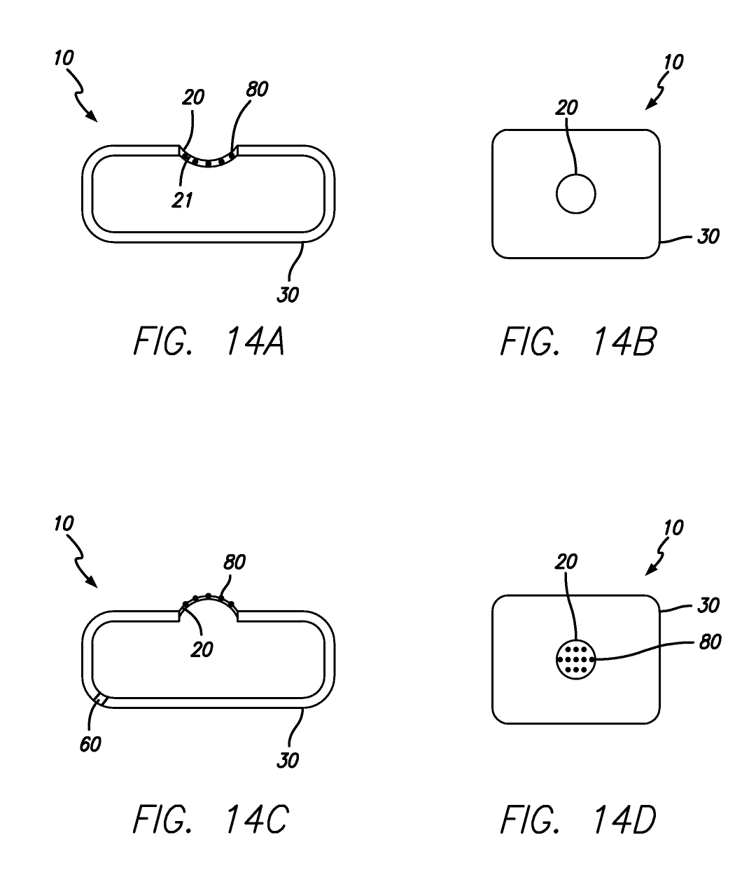

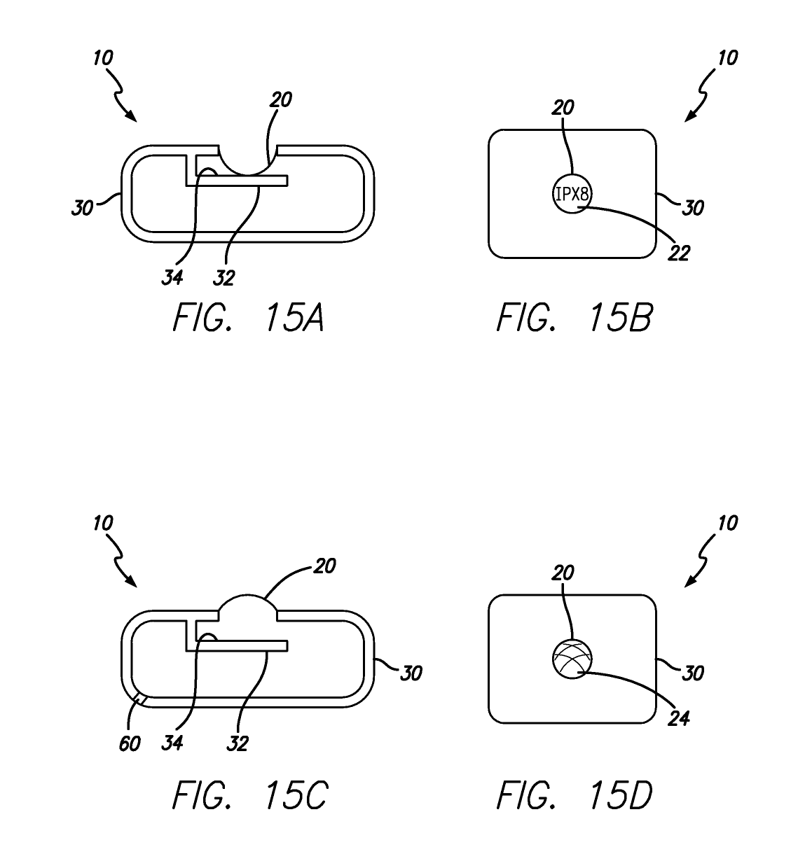

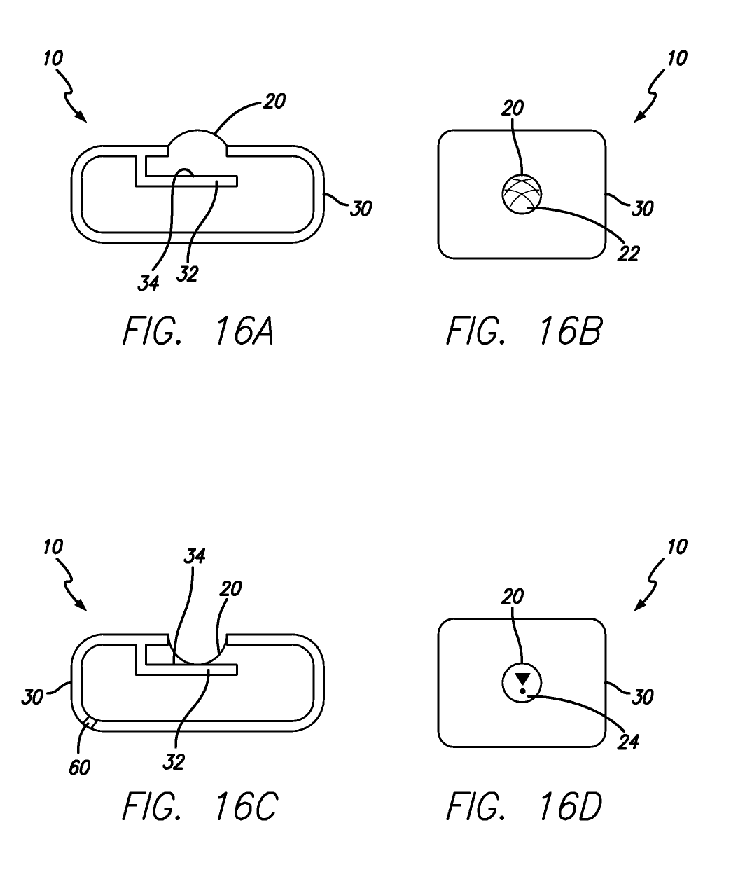

In particular embodiments shown in FIGS. 15A-15D and FIGS. 16A-16D, a portion 32 of the rigid medical device housing 30 is located below the indicator 20. In some embodiments, the portion 32 of the medical device housing 30 below the moveable indicator 20 is rigid and at least partially in contact with the indicator 20 when the indicator 20 is in a recessed position. The top surface 34 of the portion 32 of the medical device housing 30 can include a color, pattern, text, symbol, or the like. In such embodiments, the indicator 20 can be made of a translucent material such that an individual can see the color, pattern, text, symbol, or the like on the top surface 34 of the portion 32 of the medical device housing 30 when the indicator 20 is in a recessed position and/or in contact with the top surface 34 of the portion 32 of the housing 30. When the indicator 20 is in the raised position, the translucent material of the indicator 20 obstructs the view of the color, pattern, text, symbol, or the like on the top surface 34 of the portion 32 of the medical device housing 30 beneath the indicator 20. Depending on the position of the indicator 20, an individual can see the color, image, pattern, text, symbol, or the like on the top surface 34 of the portion 32 of the housing 30 when the indicator 20 at least partially contacts the top surface 34 and cannot see the color, image, pattern, text, symbol, or the like on the top surface 34 of the portion 32 of housing 30 when the indicator 20 is in a raised position.

Furthermore, in these embodiments, the portion 32 of the medical device housing 30 is structured such that it does not prevent the indicator's function to detect whether or not the housing 30 is sealed. In embodiments, the portion 32 of the medical device housing 30 beneath the indicator does not fully isolate the indicator from the interior volume 40 of the medical device 10. For example, at least one side of the portion 32 (a front, back, left, and/or right side) opens to the interior volume 40. In embodiments shown in the cross-sectional views of FIGS. 15A, 15C, 16A, and 16C, the front, back, and right sides of a portion 32 of the housing beneath the indicator 20 open to the interior volume 40 of the housing 30. Thus, the indicator 20 can still detect any change in pressure in the interior volume 40 when a leak is detected, that allows the indicator 20 to shift from a first position 22 to a second position 24 and therefore indicate a leak 60 is detected. In embodiments where the indicator 20 and housing 30 are comprised of the same materials, both the indicator 20 and the housing 30 can be made of a translucent material.

FIGS. 15A-15D illustrate a particular embodiment when the indicator 20 is in a first (recessed) position 22 in FIGS. 15A and 15B to indicate that the medical device housing 30 is sealed and a second (raised) position 24 in FIGS. 15C and 15D to indicate that the housing 30 is not sealed. For example, the housing 30 may have been sealed under low pressure or high temperature and then returned to the ambient environment to provide the first (recessed) position 22 in this embodiment in FIG. 15A. In the particular embodiment shown in FIG. 15B, as long as the housing 30 is sealed, the indicator 20 remains in the first (recessed) position 22. When the indicator 20 is in the first position 22, the recessed translucent indicator 20 is at least partially in contact with the top surface 34 of the portion 32 of the medical device housing 30 having text that reads “IPX8” to visually indicate to an individual that the housing 30 has an IPX8 waterproof seal. Once the waterproof seal is broken, the indicator 20 will shift to a second (raised) position 24, as shown in the embodiment in FIG. 15C. The text “IPX8” can no longer be seen through the raised, translucent indicator 20, as shown in the embodiment in FIG. 15D. Though the text “IPX8” is shown in the illustrated embodiment, any text, symbol, color, pattern, or the like may be utilized.

FIGS. 16A-16D illustrate alternative embodiments in which the indicator 20 is in a first (raised) position 22 in FIGS. 16A and 16B to indicate that the medical device housing 30 is sealed and a second (recessed) position 24 in FIGS. 16C and 16D to indicate that the housing 30 is not sealed. For example, the housing 30 may have been sealed under high pressure or low temperature and then returned to the ambient environment to provide the first (raised) position 22, shown in the embodiment in FIG. 16A. In the particular embodiment shown in FIG. 16B, as long as the housing 30 is sealed, the indicator 20 remains in the first (raised) position 22. When the indicator 20 is in the first position 22, any color, text, symbol, pattern or the like on the top surface 34 of the portion 32 of the housing 30 beneath the translucent indicator 20 cannot be seen through the raised indicator 20. Once the waterproof seal is broken, the indicator 20 will shift to a second (recessed) position 24 in the embodiment shown in FIG. 16C. In this embodiment, the recessed translucent indicator 20 is at least partially in contact with the top surface 34 of the portion 32 of the medical device housing 30 having a symbol “!”. As illustrated by the top view of the embodiment in FIG. 16D, the symbol “!” can now be seen through the recessed, translucent indicator 20 to provide a warning signal that the housing 30 has lost its seal. Though the symbol “!” is shown in the illustrated embodiment, any text, symbol, color, pattern, or the like may be utilized.

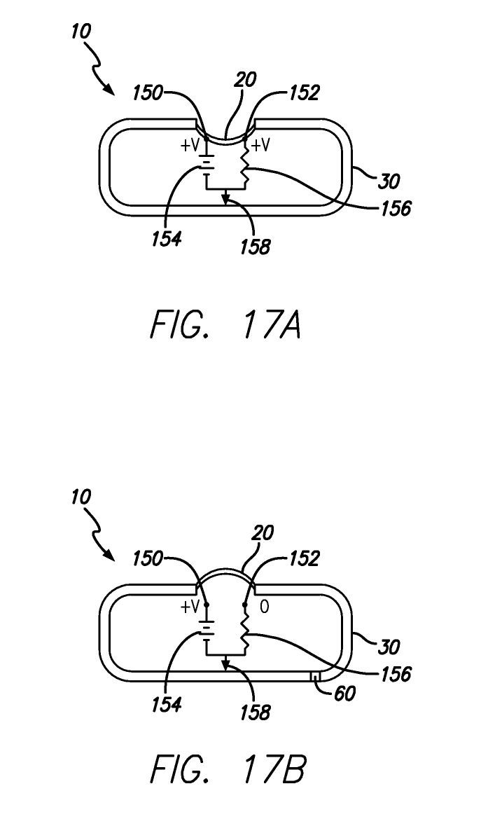

In further embodiments shown in FIGS. 17A-17B, the housing 30 can include a first electrical contact 150 and a second electrical contact 152. In such embodiments, the indicator 20 can include a conductive material. For example, embodiments of the indicator 20 may include a metal material, a metal foil, sputtered metal on the indicator, or other suitable metalized materials to make the indicator conductive. In embodiments, the first electrical contact 150 can be connected to a power source or battery 154. When the indicator 20 is in a recessed position, the conductive indicator 20 comes in contact with both the first electrical contact 150 and the second electrical contact 152 and makes the electrical contacts the same potential +V (i.e. closed circuit). Once the indicator 20 shifts to a raised or flat position, the indicator 20 no longer contacts the electrical contacts 150 and 152, loses the electrical connection, and forms an open circuit. When an open circuit, current will discharge from the second electrical contact 152, through a resistor 156, and into an electrical ground 158. The resulting potential of the second electrical contact 152 would change from +V to 0 (i.e. electrical ground). Accordingly, depending on whether the electrical contacts 150 and 152 are in contact with the indicator 20, an electrical indication of the voltage on the second electrical contact 152 can provide the position of the indicator 20, i.e., whether the indicator 20 is in a raised or flat position, or in a recessed position.

In alternative embodiments, the indicator 20 may contact the electrical contacts 150 and 152 when the indicator 20 is in a flat position. In such embodiments, the indicator 20 will lose the electrical connection with the electrical contacts 150 and 152 when the indicator is in a raised position. Accordingly, depending on whether the electrical contacts 150 and 152 are in contact with the indicator 20, an electrical indication of the voltage on the second electrical contact 152 can provide the position of the indicator 20, i.e., whether the indicator 20 is in a flat position or a raised position.

The electrical contacts can include a wired or wireless connection to a controller or processor in the medical device housing. The electrical contacts can thereby provide or transmit the information of the status of the medical device seal, based on the indicator's position, to a medical device controller or processor. In some embodiments, the device can provide a sensory signal, such as an auditory signal or a visual signal on a display 102, when the indicator has moved from a first position to a second position, or vice versa, when the indicator has moved from a second position to a first position.

In further embodiments, a medical device processor may record and store the seal status information in a memory. In particular embodiments, the time of the seal break may be recorded and/or provided. In yet further embodiments, the processor may provide the information to the user via a display 102 on the medical device 10. In other embodiments, the processor may provide or transmit the information to a remote device such as a computer, smartphone, or the like. In other embodiments, the device 10 can automatically shut itself down if the processor receives a signal that indicates the medical device seal is broken via the detection of the indicator 20 position. In further embodiments, an individually can manually shut down the device 10 if notified via the indicator 20, a display 102, or a remote device that the seal has been broken.

In yet further embodiments, the indicator 20 can provide a sound when the indicator 20 moves from the first position 22 to the second position 24. For example, the indicator 20 can make a popping or clicking sound when moving from the first position 22 to the second position 24.

In alternative embodiments, the indicator 20 can be utilized with one or more components of a medical device system such as an infusion system. In some embodiments, an infusion system can include an infusion pump 100 to deliver an agent, such as insulin or another prescribed medication, therapeutic agent, or the like to an individual. A typical infusion pump includes a pump drive system which generally includes a small motor and drive train components that convert rotational motor motion to a translational displacement of a plunger (or stopper) in a reservoir. The pump delivers medication from the reservoir to the body of a user via a fluid path created between the reservoir and the body of a user. For example, the medication may flow from the reservoir, through a tubing, and then through a cannula or needle into the individual's body. In alternative embodiments, the infusion pump may be worn on the individual's body or directly adhere to an individual's skin to deliver a prescribed medication. It is therefore important that the components within the interior volume 40 of the infusion pump housing 30 are enclosed by a sealed housing 30 to protect the components from water damage or the like.

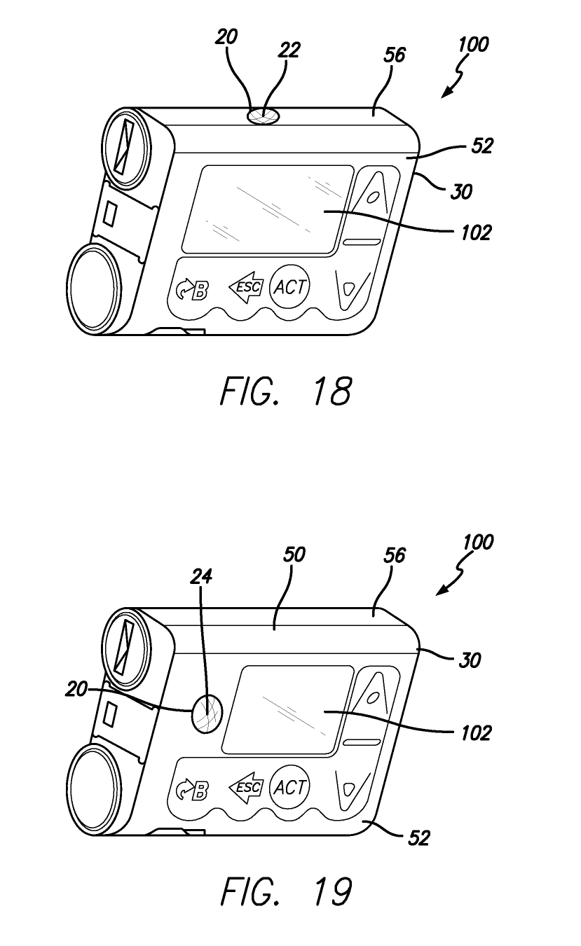

As shown in the embodiments in FIGS. 18 and 19, the indicator 20 could be incorporated in the housing 30 of an infusion pump 100. FIG. 18 shows an infusion pump 100 having an indicator 20 on an exterior side surface 56 of the infusion pump housing 30. In this embodiment, the indicator 20 is in a recessed first position 22 and can indicate that the infusion pump 100 has a proper seal and is waterproof. In the embodiment shown in FIG. 19, the indicator 20 is located on a front exterior surface 52 of the infusion pump housing 30. In this embodiment, the indicator 20 is in a raised second position 24 and can indicate that the infusion pump 100 is not sealed and not waterproof.

Examples of infusion pumps that can incorporate the indicator as described herein include, but are not limited to Medtronic MiniMed, Inc. products such as Minimed® 530G system, Paradigm® insulin pump, and other external or on-body patch type infusion pumps. Further examples of infusion pumps 100 used to administer insulin or other medications may be of the type described in, but not limited to, U.S. Pat. Nos. 4,562,751; 4,678,408; 4,685,903; 5,080,653; 5,097,122; 5,505,709; 6,485,465; 6,551,276; 6,554,798; 6,558,320; 6,558,351; 6,641,533; 6,659,980; 6,752,787; 6,817,990; 6,872,200; 6,932,584; 6,936,029; 6,979,326; 6,997,920; 7,025,743; 7,109,878; 7,402,153; 7,621,893; and 7,819,843, which are herein incorporated by reference. That said, the subject matter described herein is not limited to infusion devices and may be implemented in an equivalent manner for any medical device capable of regulating, monitoring or otherwise influencing a condition of an associated user that wears or otherwise operates the medical device on his or her body.

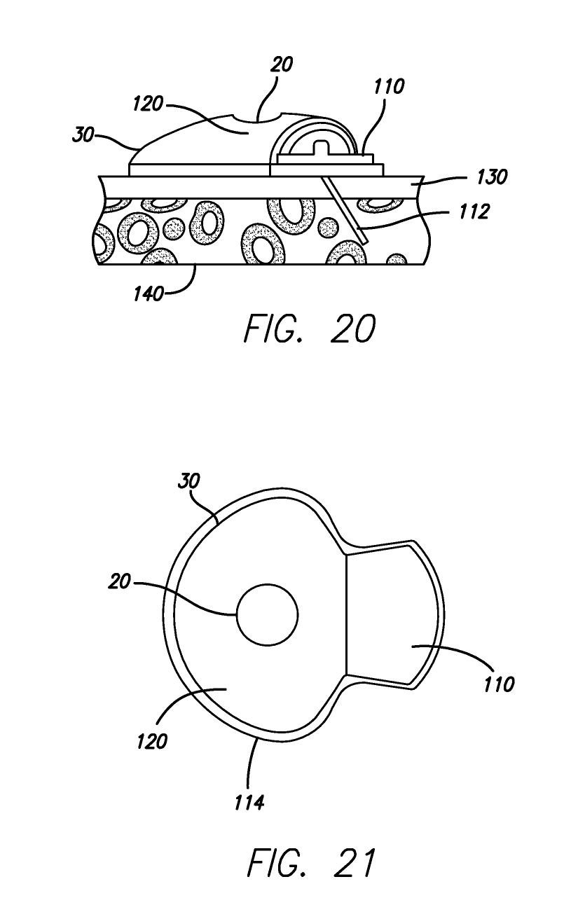

The infusion system can include a sensor 110 and transmitter 120 to measure and/or monitor a physiological condition of an individual and send the measured data to another component of a medical system, such as the infusion pump 100. As a non-limiting example, the sensor 110 may be a glucose sensor to measure glucose levels in the body. The transmitter 120 can connect to the sensor 110 and send the glucose data to an insulin infusion pump 100. The insulin pump can receive and display the glucose readings as well as deliver insulin to the individual's body. In the embodiment shown in FIG. 20, the sensor 110 and transmitter 120 are directly adhered to the individual's body. In embodiments, the sensory component can be incorporated an element 112 such as a cannula, needle, or the like to penetrate past the individual's skin 130 into the interstitial fluid 140 to measure a physiological condition. As illustrated in FIG. 21, the sensor 110 and/or transmitter 120 can include an adhesive layer 114 to attach to the individual's skin 130.

Also shown in the embodiments of FIGS. 20 and 21, an indicator 20 can be incorporated in the housing 30 of the transmitter 120. The indicator 20 provides a benefit to these embodiments of the invention utilizing portable medical devices 10 or related components, such as transmitters, that are worn on the individual user's body in a continuous manner. In such embodiments, the indicator 20 can detect and indicate a leak 60 in the medical device 10 after the device 10 is worn on the body and exposed to, or submersed in, water (while swimming, bathing, showering or the like).

As non-limiting examples, the indicator could be incorporated in the housing of a continuous glucose measurement or monitoring system, including, but not limited to Medtronic MiniMed, Inc. products such as Sof-Sensor®, Enlite®, iPro®, and MiniLink® transmitter. The apparatus could also be used with sensor and sensor transmitters generally described by way of example in U.S. Pat. Nos. 5,586,553; 6,248,067; 6,809,653; and 8,550,997, the disclosures of which are herein incorporated by reference in their entireties.

By incorporating the indicators 20 into medical devices 10, manufacturers of the medical devices could screen the devices for proper seals and defective seals before the devices leave the manufacturing site. In some cases, the device manufacturers may be able to provide a waterproof rating without water immersion testing. Thus, the indicator 20 can streamline manufacturing and reduce associated time, costs, and resources of the manufacturing process. The indicator 20 provides a safety warning that the device is no longer waterproof to the end user as well. Once the device is in the hands of the user, the user can easily observe by the position of the indicator 20 to determine if the waterproof seal is broken and possibly return the device 10 to the manufacturer for repair.

Different methods for determining if a medical device housing 30 has a waterproof seal utilizing an indicator 20 can be ascertained from the aforementioned embodiments of the invention described herein. For example, in one embodiment, the method can comprise: providing a medical device housing 30 having an interior volume 40, an exterior surface 50, and an indicator 20 on the exterior surface 50, where the indicator 20 is moveable from a first position 22 to a second position 24; sealing the medical device housing 30 in a low pressure environment; returning the medical device housing 30 to a normal atmospheric environment such that the indicator 20 moves to the first position 22, wherein the indicator 20 remains in the first position 22 so long as the seal is intact and wherein the indicator moves to the second position 24 when there is a leak 60 in the housing. The user of the device 10 can then observe or feel the position of the indicator 20 to determine whether the medical device housing 30 is sealed.

In another embodiment, a method for determining whether a medical device housing 30 is sealed can generally comprise providing a sealed medical device housing 30 having an interior volume 40, an exterior surface 50, and an indicator 20 on the exterior surface 50. The indicator 20 is moveable from a first position 22 to a second position 24 and the indicator 20 is in the first position 22. Once the medical device is provided, an individual may observe or feel the position of the indicator 20 to determine whether the medical device housing 30 is sealed. The first position 22 can indicate that the medical device housing 30 is sealed and the second position 24 can indicate that there is a leak 60 in the medical device housing 30. In embodiments, the step of providing the sealed medical device housing 30 can further include sealing the medical device housing 30 in a low pressure or high temperature environment, or a combination of a low pressure and high temperature environment. In alternative embodiments, the step of providing the sealed medical device housing 30 can further include sealing the medical device housing 30 in a high pressure or low temperature environment, or a combination of a high pressure and low temperature environment.

While the description above refers to particular embodiments of the present invention, it will be understood that many modifications can be made without departing from the spirit thereof. The accompanying claims are intended to cover such modifications as would fall with the true scope and spirit of the present invention. The presently disclosed embodiments are therefore to be considered in all respects as illustrative and not restrictive, the scope of the invention being indicated by the appended claims, rather than the foregoing description, and all changes within the meaning and range of equivalency of the claims are therefore intended to be embodied therein.

What is claimed is:

1. A medical device having an indicator to indicate whether the medical device is sealed, the medical device comprising:
a medical device housing having an interior volume, an exterior surface, and the indicator on the exterior surface of the medical device housing, the indicator capable of moving from a first position to a second position, wherein the first position indicates that the medical device housing has a seal to prevent ingress into the interior volume of the medical device and the second position indicates a leak in the housing.

2. The medical device of claim 1, wherein the first position protrudes inward toward the interior volume of the medical device housing.

3. The medical device of claim 2, wherein the second position protrudes outward from the exterior surface of the medical device housing and away from the interior volume of the medical device housing.

4. The medical device of claim 2, wherein the second position is flush with the exterior surface of the medical device housing.

5. The medical device of claim 1, wherein the first position is flush with the exterior surface of the housing; and
the second position protrudes outward from the exterior surface of the medical device housing and away from the interior volume of the medical device housing.

6. The medical device of claim 1, wherein the exterior surface of the housing is rigid and the indicator is flexible.

7. The medical device of claim 1, wherein the medical device is an infusion pump.

8. The medical device of claim 1, wherein the medical device is a transmitter.

9. The medical device of claim 1, wherein the seal is a waterproof seal.

10. The medical device of claim 1, wherein the indicator provides a sound when the indicator moves from the first position to the second position.

11. The medical device of claim 1, wherein the indicator further includes at least one tactile element, the at least one tactile element protrudes from the indicator when the indicator is in the second position.

12. The medical device of claim 1, the indicator further including a first pattern displayed in the first position and a second pattern displayed in the second position.

13. The medical device of claim 1, the medical device housing further including a portion located beneath the indicator, wherein a top surface of the portion of the medical device housing located beneath the indicator includes at least one of a color, pattern, symbol and text; and
wherein the indicator is translucent such that at least one of the color, pattern, symbol and text on the top surface of the portion of the medical device housing located beneath the indicator is visible through the indicator only when the indicator at least partially contacts the top surface of the portion of the housing located beneath the indicator.

14. The medical device of claim 1, the medical device housing further including a first electrical contact and a second electrical contact;
wherein the indicator further includes a conductive material; and
wherein the indicator contacts the first electrical contact and the second electrical contact only when the indicator is in the first position to provide an electrical indication of whether the indicator is in the first position or the second position and thereby indicate whether the medical device is sealed.

15. The medical device of claim 1, the indicator further including a first color displayed in the first position and a second color displayed in the second position.

16. The medical device of claim 1, wherein the indicator includes a polymeric material.

17. A method for determining whether a medical device housing is sealed, the method comprising the steps of:
providing a sealed medical device housing having an interior volume, an exterior surface, and an indicator on the exterior surface, wherein the indicator is in a first position and the indicator is moveable from the first position to a second position; and
observing the position of the indicator to determine whether the medical device housing is sealed, wherein the first position indicates that the medical device housing is sealed and the second position indicates that there is a leak in the medical device housing.

18. The method of claim 17, wherein the step of providing the sealed medical device housing further includes at least one of: overmolding the indicator on the exterior surface of the housing; ultrasonically welding the indicator on the exterior surface of the housing; adhesively bonding the indicator on the exterior surface of the housing; vacuum forming the indicator on the exterior surface of the housing; blow molding the indicator on the exterior surface of the housing; injection molding the housing and indicator; and 3-D printing the housing and indicator.

19. The method of claim 17, wherein the step of providing the sealed medical device housing further includes at least one of: sealing the medical device housing in a low pressure environment; sealing the medical device housing in a high temperature environment; sealing the medical device housing in a low pressure and a high temperature environment; sealing the medical device housing in a high pressure environment; sealing the medical device housing in a low temperature environment; and sealing the medical device housing in a high pressure and a low temperature environment.

20. A method for determining whether a medical device housing is sealed, the method comprising the steps of:
providing a medical device housing having an interior volume, an exterior surface, and an indicator on the exterior surface, the indicator is moveable between a first position and a second position, wherein the first position protrudes inward toward the interior volume of the medical device housing and the second position protrudes outward from the exterior surface of the medical device housing and away from the interior volume of the medical device housing;
sealing the medical device housing in at least one of a low pressure environment and a high temperature environment, wherein the indicator is in the second position during the step of sealing the medical device housing; and
returning the medical device housing to a normal atmospheric environment such that the indicator moves from the second position to the first position, wherein the indicator remains in the first position so long as a seal is intact and wherein the indicator moves to the second position when there is a leak in the medical device housing.
